(19) United States
(12) Patent Application Publication
(10) Pub. No.: US 2010/0172853 A1
(43) Pub. Date: Jul. 8, 2010

(54) COSMETIC COMPOSITIONS CONTAINING A NATURALLY-OCCURRING POLYPEPTIDE FILM FORMER

(75) Inventors: Florentina Pavel, Hillsborough, NJ (US); Lila Patel, Edison, NJ (US); Bruno Bavouzet, Hoboken, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 12/645,787

(22) Filed: Dec. 23, 2009

Related U.S. Application Data
(60) Provisional application No. 61/204,098, filed on Dec. 31, 2008.

(51) Int. Cl. A61K 8/64; A61K 8/92; A61Q 1/10

(52) U.S. Cl. 424/63; 514/2; 514/12; 514/8; 424/70.7

(57) ABSTRACT

Disclosed are cosmetic compositions containing naturally-occurring polypeptide film-former, such as zein, a thickener, and a solvent. Also disclosed are methods for applying such cosmetic compositions to keratinous tissues, such as skin or eyelashes, in order to enhance their appearance and methods of making such compositions.

### CROSS-REFERENCE TO RELATED APPLICATIONS

This application claims the benefit of the filing date of U.S. Provisional Patent Application No. 61/204,098 filed Dec. 31, 2008, the disclosure of which is hereby incorporated herein by reference.

### BACKGROUND OF THE INVENTION

Cosmetic compositions may contain various film-forming polymers. U.S. Pat. No. 7,351,418 teaches cosmetic compositions, such as mascara, that contain a polymer containing a specific heteroatom in a composition comprising particles of a film-forming polymer dispersed in the composition medium, which improves adhesion properties and allows for a thick deposit of the composition on the keratin materials.

U.S. Pat. No. 7,351,405 teaches wax-free mascara compositions containing solid particles of a film-forming acrylic polymer dispersed in a cosmetically acceptable aqueous medium.

U.S. Pat. No. 6,641,823 teaches cosmetic compositions, such as eyeliners, that contain aqueous medium, at least one film-forming polyethylene glycol/polypropylene glycol/polyethylene glycol triblock polycondensate polymer, at least one nonionic surfactant, and at least one pulverulent coloring matter.

U.S. Pat. No. 6,641,821 teaches cosmetic compositions, including eyeliners, containing a fat-soluble film-forming polymer, a volatile oil and a polyolefin wax.

### SUMMARY OF THE INVENTION

Underlying the present invention is the discovery that the use of a naturally-occurring polypeptide film former, such as a plant-derived polypeptide, provides cosmetic compositions having improved wear properties.

A first aspect of the present invention is directed to a cosmetic composition containing a naturally-occurring polypeptide film-former, a thickener, and a solvent. In some embodiments, the polypeptide may be plant-derived and meet organic standards. Also disclosed are packages, e.g., blister packages, including a container containing the cosmetic composition, and optionally an applicator for applying the composition to keratinous tissue.

A second aspect of the present invention is directed to a method of making up or enhancing the appearance of keratinous tissue, by applying to keratinous tissue a cosmetic composition containing a naturally-occurring polypeptide film-former, a thickener, and a solvent.

A third aspect of the present invention is method of preparing or making a cosmetic composition by mixing together a naturally-occurring polypeptide film-former, a thickener, and a solvent.

As illustrated in the working examples herein, an embodiment of the present invention exhibited unexpectedly greater wear, including less flaking, compared to mascara compositions containing a conventional non-proteinaceous film former or no film former. In addition to the polypeptide, the inventive cosmetic compositions can be made entirely from organic-grade ingredients, thus allowing for substantially environmentally friendly products and processes.

### DETAILED DESCRIPTION

The naturally-occurring polypeptide film-formers useful in the present invention are polypeptides that are cosmetically acceptable, may be dissolved in a cosmetically acceptable solvent, and form a film upon drying. A polypeptide is any combination of two or more amino acids, such as a dipeptide, tripeptide, or a protein.

The polypeptides useful in the present invention are naturally-occurring. As used herein, “naturally-occurring” refers to a substance that exists in nature or is readily obtainable (e.g., extractable or transformable) from a naturally-occurring material. Sources of the polypeptides include plants, minerals, animal products, such as beehive products and silk, fungi, yeasts, protists, and prokaryotes. Polypeptides from these materials may be extracted or purified by standard processes including crushing, grinding, desiccation, pressing, distillation, extraction (with natural solvents, water, or carbon dioxide), centrifugation, filtration, purification, ionic exchange processes, and preservations. In addition, naturally-occurring polypeptides may be obtained from these materials by straightforward or relatively simple transformations including alkylation, amide formation and polyamidification, calcination, condensation and addition, glucosylation and glycosylation, etherification, hydration, hydrogenation, hydrolysis, neutralization, oxidation and reduction, carbonation and polycarbonation, metathesis, dissolution and precipitation, cycloaddition, and photochemical reaction.

In an embodiment of the present invention, the polypeptide is derived from a plant grown under organic standards and is produced or processed under organic standards. Organic standards vary, but a representative example of such standards is that from EcoCert® (L′Isle Jourdain, France), which requires that at least 95% of the ingredients are natural (including water), at least 95% of all plant material are from organic agriculture, and that at least 10% of the ingredients are certified as organic. Organic standards have also been set forth by the U.S. Department of Agriculture, the European Union, and Japan.

In certain embodiments, the polypeptide is plant-derived. The term “plant-derived” means that the polypeptide is obtainable from a plant or plant product. In certain embodiments of the cosmetic compositions of the present invention, the polypeptide is derived from a grain or cereal, such as corn, wheat, rye, or barley. In some embodiments, the polypeptide is a prolamine. Prolamines are a group of globulin proteins having a high proline content found in grasses, most prominently cereal crops such as corn, wheat, rye, barley. Prolamines are soluble in dilute alcohol, acids, and alkali, but insoluble in water, absolute alcohol, and neutral salt solutions. In one embodiment, the prolamine is gliadin, hordein, or zein, which is commercially available from Freeman Industries, LLC (Tuckahoe, N.Y.) under the trade names Zein F4000, Zein F4400, and Zein F6000. Gliadin is a prolamine derived from wheat or rye. Hordein is a prolamine derived from barley. Zein is a prolamine derived from corn. The polypeptide may also be “animal-derived,” meaning that the polypeptide is obtainable from an animal or animal product.

The polypeptide is present in the inventive compositions in amounts generally ranging from about 0.5% to about 50% by weight, in some embodiments from about 1% to about 40% by weight, and in yet other embodiments from about %5 to about 30% by weight, based on the total weight of the composition. In an embodiment, the polypeptide is present in the inventive compositions in amounts generally ranging from about 0.5% to about 20% by weight, in some embodiments from about 1% to about 10% by weight, and in yet other embodiments from about %3 to about 7% by weight, based on the total weight of the composition. In another embodiment, the polypeptide is present in the inventive compositions in amounts generally ranging from about 10% to about 50% by weight, in some embodiments from about 20% to about 40% by weight, and in yet other embodiments from about %25 to about 35% by weight, based on the total weight of the composition.

The thickeners useful in the compositions of the present invention generally include those that thicken or gel an aqueous phase and those that thicken or gel a liquid fatty phase. Suitable thickeners for a water phase include polysaccharides and gums, e.g., natural gums such as guar gum, for example, TICorganic® Guar 3500, commercially available from TIC Gums, Inc. (White Marsh, Md.), xanthan gum, for example, Rhodicare XC, commercially available from Rhodia Novecare (Cranbury, N.J.), sclerotium, carrageenan and pectin; polysaccharide resins such as starch and its derivatives, for example tapioca starch, commercially available from National Starch, Co. (Bridgewater, N.J.) under the trade name Naviance™ tapioca, polyvinylpyrrolidone (PVP), polyvinyl alcohol, crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; hyaluronic acid and its salts, clays, and, in particular, montmorillonites, hectorites, bentonites, and laponites, crosslinked polyacrylic acids, such as the “Carbopol” products from the company Goodrich, the polyglyceryl (meth)acrylates polymers sold under the names “Hispagel” or “Lubragel” by the companies Hispano Quimica or Guardian, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare S.C.95” by the company Allied Colloid, and associative polymers and, in particular associative polyurethanes.

Representative thickeners for a fatty or oil phase include those in polymeric form and those in mineral form. The thickener may cause gelling via chemical reticulation and agents that gel via physical reticulation. Modified clays may be used as thickeners, including hectorites modified with an ammonium chloride of a C₁₀ to C₂₂ fatty acid, such as hectorite modified with distearyldimethylammonium chloride, also known as quaternium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as stearalkonium bentonites, such as the products sold or made under the names Claytone APA and Claytone AF by the company Southern Clay, and Baragel 24 sold or made by the company Rheox. Other mineral thickeners include silica, such as fumed silica. The fumed silica may have a particle size ranging from about 5 nm to 200 nm.

The thickener is present in the inventive compositions in amounts generally ranging from about 0.01% to about 10% by weight, in some embodiments from about 0.1% to about 5% by weight, and in yet other embodiments from about 0.2% to about 1% by weight, based on the total weight of the composition. In a particular embodiment, the thickener is present in an amount from about 0.2 to about 0.7%.

Typically, the solvent in the cosmetic compositions of the present invention include water. Water is present in the compositions of the present invention in amounts generally ranging from about 0.1% to about 99% by weight, in some embodiments about 10% to about 80%, and in yet other embodiments about 30% to about 60%.

Other, non-aqueous solvents, such as, polar or non-polar, or volatile or non-volatile solvents, may be present in the cosmetic compositions of the present invention, depending on the nature of the polypeptide film-former and/or other ingredients therein. Representative volatile solvents include polar volatile solvents, examples of which include lower alkanols, e.g., C₂ to C₅ alcohols, such as ethanol, ethyl 3-ethoxypropionate and isohexyl neopentanoate. Non-polar volatile hydrocarbon-based oils (which as used herein, refers to oil containing only hydrogen and carbon atoms) may also be used, silicone oils (optionally comprising alkyl or alkoxy groups that are pendant or at the end of a silicone chain), and fluoro oils. Suitable hydrocarbon-based oils include isoparaffins, i.e., branched alkanes containing 8-16 carbon atoms, such as isododecane (also known as 2,2,4,4,6-pentamethylheptane), and petroleum distillates. Suitable silicone oils may include linear or cyclic silicones containing from 2 to 7 silicon atoms, these silicones optionally comprising alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane and heptamethyloctyltrisiloxane, and mixtures thereof. Mixtures of these solvents may be used.

The volatile solvent may be present in the cosmetic composition of the present invention in an amount generally ranging up to about 90%, in some embodiments from about 1% to about 80%, in other embodiments from about 2% to about 70%, in other embodiments from about 5% to about 50%, and in yet other embodiments from about 5 to about 10%, based on the total weight of the composition.

Representative examples of non-polar non-volatile solvents include polyalphaolefins, which include ethylene derivatives oligomerized into even-numbered carbon polyalphaolefins e.g., C₆-C₁₄ olefins such as polydecene and polymers of C₆, C₈, C₁₂ and C₁₄ olefins. The polyolefins may have a molecular weight (MW) generally ranging from about 280 to about 11,500, and a viscosity (CPs at about 20° C.) generally ranging from about 7 to about 32,500. They may also be hydrogenated. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively.) PureSyn™ 100 (MW about 2939, viscosity about 3900, INCI name: hydrogenated C₆₋₁₄ olefin polymers) and PureSyn™ 1000 (MW about 11,500, viscosity about 32,400, INCI name: polydecene) may also be useful. The PureSyn™ products are available from Exxon Chemicals.

A non-volatile solvent may be present in the cosmetic composition of the present invention in an amount generally ranging from about 0.1% to about 70%, and in some embodiments, about 0.5% to about 40%, and in other embodiments, 1% to about 25%, based on the total weight of the composition.

One or more water-miscible solvents (miscibility in water of greater than 50% by weight at about 25° C.) may also be present. Examples include lower monoalcohols containing from 1 to 5 carbon atoms such as ethanol and isopropanol, glycols containing from 2 to 8 carbon atoms, such as propylene glycol, ethylene glycol, butylene glycol or dipropylene glycol and pentylene glycol, C₃-C₄ ketones and C₂-C₄ aldehydes.

In certain embodiments of the present invention the naturally-occurring polypeptide film-former is a prolamine. In these embodiments, the composition contains a lower alkanol, such as ethanol, and water. Typically, the alkanol is present in an amount, by weight, about equal to the amount of prolamine, by weight, based on the total weight of the composition.

The compositions of the present invention may contain additional (e.g., cosmetically or dermatologically acceptable) ingredients, such as waxes, oils, emulsifiers, colorants, dispersion enhancing agents, other polymers or film-formers, moisturizers, fibers, pH adjusters, fillers, powders, mothers-of-pearl, preservatives, plasticizers, and detackifiers.

For the purposes of the present invention, the term “wax” means a lipophilic fatty compound that is solid at room temperature about (25° C.) and atmospheric pressure (760 mmHg, i.e., 105 Pa), which undergoes a reversible solid/liquid change of state and which has a melting point of greater than 30° C. and in some embodiments, greater than about 55° C. up to about 120° C. or even as high as about 200° C. For the purposes of the invention, the waxes are those generally used and acceptable in cosmetics and dermatology. A variety of waxes may be useful, including waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fibre waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes. Alternatively, hydrogenated oils of animal or plant origin may be used. Examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils. In some embodiments, the compositions contain at least two or at least three waxes. The wax may be present in the compositions in an amount generally ranging from about 0.1% to about 40%, and in some embodiments about 0.5% to about 20%, or about 1% to about 10% by weight, relative to the total weight of the composition.

Examples of cosmetically or dermatologically acceptable oils include carbon-based, hydrocarbon-based, fluoro and/or silicone oils, of mineral, animal, plant or synthetic origin, alone or as a mixture. A representative example of an oil of plant origin useful in the present invention is argania spinosa kernel oil, commercially available from Alban Muller International (Vincennes, France) and from DiaSouss GmbH (Munich, Germany) as Argan Oil. These ingredients, in addition to any other non-polar ingredients, would constitute a liquid fatty phase or oil phase of the cosmetic composition.

In those embodiments wherein the cosmetic composition contains water, and an oil phase, the compositions of the invention may also contain an emulsifier. Emulsifiers typically employed in the compositions of the present invention include anionic, nonionic and cationic emulsifiers. See, e.g., Encyclopedia of Chemical Technology, KIRK-OTHMER, volume 22, pp. 333-432, 3rd edition, 1979, Wiley, for the definition of the properties and (emulsifying) functions of the emulsifiers, in particular pp. 347-377 of this publication regarding anionic and nonionic emulsifiers. Examples of emulsifiers useful in the compositions of the invention include as nonionic emulsifiers, fatty acids, such as palmitic acid, fatty alcohols, including C₁₂ to C₁₈ fatty alcohols, such as cetyl alcohol, stearyl alcohol, and cetearyl alcohol, polyethoxylated fatty alcohols or polyglycerolated fatty alcohols, such as polyethoxylated stearyl alcohols or cetylstearyl alcohols, esters of fatty acid and sucrose, esters of fatty acids and glycerin, such as glyceryl stearate, and glucose alkyl esters, in particular cetearyl glucoside and polyoxyethylenated C₁-C₆ alkyl glucose fatty esters. A mixture of anionic emulsifiers may be used, such as a mixture of 80% cetearyl alcohol and 20% cetearyl glucoside, commercially available from SEPPIC S.A. (Paris, France) under the trade name MONTANOV® 68. Representative examples of anionic emulsifiers useful in the compositions of the present invention include, C₁₆-C₃₀ fatty acids neutralized by amines, ammonia or the alkali metal salts thereof, and salts of acylaminoacids, such as sodium stearoyl glutamate, commercially available from Anjinomoto U.S.A., Inc. (Fort Lee, N.J.) under the trade name AMISOFT® HS 11 PF. Examples of cationic emulsifiers include quaternary amines, amine oxides and amines, e.g., alkyl amines, alkyl imidazolines, ethoxylated amines, quaternary compounds, and quaternized esters. Cationic emulsifiers may also provide a conditioning effect.

Emulsifiers are generally present in amounts ranging from about 1 to about 30% by weight, and in some other embodiments from about 3% to about 15% by weight, relative to the total weight of the composition.

The inventive compositions may also contain a colorant. Colorants may be chosen from the lipophilic dyes, hydrophilic dyes, traditional pigments, and nacres usually used in cosmetic or dermatological compositions, and mixtures thereof. The coloring agent may have any shape, such as, for example, spheroidal, oval, platelet, irregular, and mixtures thereof. Pigments may optionally be surface-treated e.g., with silicones (e.g., inorganic pigments may be coated with simethicone), perfluorinated compounds, lecithin, and amino acids.

The liposoluble dyes include, for example, Sudan Red, D&C Red 17, D&C Green 6, soybean oil, Sudan Brown, D&C Yellow 11, D&C Violet 2, D&C Orange 5, quinoline yellow and annatto. The water-soluble dyes are, for example, beetroot juice or methylene blue.

The pigments may be chosen from white pigments, colored pigments, inorganic pigments, organic pigments, coated pigments, uncoated pigments, pigments having a micron size and pigments not having a micron size. Among the inorganic pigments that may be mentioned are iron oxide, titanium dioxide, optionally surface-treated, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminum.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

Colorants are generally present in an amount ranging from about 0.01% to about 50% relative to the total weight of the composition. In certain embodiments, the colorant is present in an amount of from about 0.1% to about 30%, by weight, in other embodiments from about 0.5% to about 20%, by weight, and in yet other embodiments from about 1% to about 10%, by weight, based on the total weight of the composition. In particular embodiments, the colorant is present in an amount of about 6% or about 10%, by weight.

The compositions of the present invention may also contain dispersion enhancing agents such as polysaccharide resins, e.g., KM 13, available from KAMA International Corp. (Duluth, Ga.), or a mixture of emulsifiers, such as fatty alcohols, such as C₁₂-C₁₆ fatty alcohols, fatty acids, eg., and/or phospholipids. A suitable example of a dispersion enhancing agent is a natural mixture of C₁₂-C₁₆ fatty alcohols, vegetal palmitic acid, and soy lecithin, e.g., Biophilic S PCR, commercially available from Lucas Meyer Cosmetics ZA (Versailles, France). Dispersion enhancing agents are typically employed in compositions that contain a colorant.

Dispersion enhancing agents are generally present in an amount ranging from about 0.01% to about 20% relative to the total weight of the composition. In certain embodiments, the dispersion enhancing agent is present in an amount of from about 0.1% to about 15%, by weight, and in other embodiments from about 0.2% to about 10%, by weight, and yet other embodiments from about 0.5% to about 5%, based on the total weight of the composition. In a particular embodiment, the dispersion enhancing agent is present in an amount of about 1%, by weight, based on the total weight of the composition.

The cosmetic compositions may contain other polymers or film-formers, e.g., film-forming polymers, provided that they are compatible with the other ingredients in the inventive compositions and particularly the polypeptide. The polymer may be present in the compositions in an amount generally ranging from 0.01% to about 20%, by weight, based on the total weight of the composition.

Compositions of the present invention may also contain a moisturizer. Examples include sodium lactate, mannitol, amino acids, hyaluronic acid, lanolin, urea, petroleum jelly and mixtures thereof. Other examples include polyols such as glycerin, diglycerin, triglycerin, polyglycerin, polyethylene glycol, ethylene glycol, diethylene glycol, triethylene glycol, propylene glycol, dipropylene glycol, hexylene glycol, 1,3-butylene glycol, 1,4-butylene glycol and sorbitol. Moisturizers may be present in the compositions of the present invention in amounts generally ranging from about 0.1% to about 20%, and in some embodiments, from about 0.5% to about 15%, by weight, based on the total weight of the composition.

In some embodiments, the cosmetic composition, for example a mascara, may further contain fibers to allow an improvement in a lash lengthening effect. The fibers useful in the present invention may be chosen from natural and synthetic fibers. Representative examples of natural fibers include, cotton, silk, wool, and other keratin fibers. Representative examples of synthetic fibers include, polyester, rayon, nylon and other polyamide fibers. The fibers may be present in the compositions in an amount generally ranging from about 0.01% to about 10%, by weight, based on the total weight of the composition.

The cosmetic compositions of the present invention may contain a pH adjuster. Representative examples of pH adjusters include organic acids such as citric acid, malic acid, lactic acid, succinic acid and oxalic acid, sodium salts of the acids, and alkaline agents such as ammonia, monoethanolamine, diethanolamine, triethanolamine, sodium hydroxide, potassium hydroxide, sodium carbonate, sodium hydrogencarbonate, potassium carbonate and potassium hydrogencarbonate.

The pH of the cosmetic compositions of the present invention may vary, e.g., from moderately acidic to moderately alkaline provided that the composition is compatible and non-abrasive to the substrate to which it will be applied. In an embodiment of the present invention, the pH of the waving composition is in the range of about 4 to about 9 and in other embodiments the pH is about 6 to about 8. In a particular embodiment, the pH of the waving composition is about 7.

Fillers, powders and mothers-of-pearl may also be present, typically to modify the texture of the composition and the matteness/gloss effect. Representative examples of these types of ingredients include mica, silica, kaolin, iron oxides, titanium dioxide, polyamide powders, polyamide powders, for instance Nylon® (Orgasol from Atochem), poly-alanine powders, polyethylene powders, tetrafluoroethylene polymer powders, for instance Teflon®, starch, boron nitride, hollow polymer microspheres such as those of polyvinylidene chloride/acrylonitrile, for instance Expancel® (Nobel Industrie), acrylic powders such as Polytrap® (Dow Corning), polymethyl methacrylates particles and silicone resin microbeads (for example Tospearls® from Toshiba), magnesium hydrocarbonate, hydroxyapatite, hollow silica microspheres (Silica Beads® from Maprecos), and glass and ceramic microcapsules. Filler(s), if present, are in amounts generally ranging from about 0.1% to about 25%, and in some embodiments from about 0.5% to about 20%, by weight, based on the total weight of the composition.

The inventive compositions may also include a preservative. Representative examples of preservatives include bezoates, such as, sodium benzoate, benzyl alcohol; alkyl para-hydroxybenzoates, wherein the alkyl radical has from 1, 2, 3, 4, 5 or 6 carbon atoms, such as, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol; sorbic acid and its salts, and dehydroacetic acid and its salts. Mixtures of preservatives are also useful, e.g., the mixture of methylparaben, ethylparaben, propylparaben and butylparaben sold under the name Nipastat by Nipa, the mixture of phenoxyethanol, methylparaben, ethylparaben, propylparaben and butylparaben, also sold by Nipa under the name Phenonip, and the mixture of phenoxyethanol, methylparaben, isopropylparaben, isobutylparaben and butylparaben, sold by ISP under the tradename Liquapar Optima. The preservative may be present in an amount generally ranging from about 0.01% to about 15%, by weight, based on the total weight of the composition.

Plasticizers useful in the present invention are compounds or compositions that, when combined with the naturally-occurring polypeptide film-former, produce a film with increased flexibility. Representative examples of plasticizers include polyols, such as glycerin; glycols, including butylene glycol, polyethylene glycol, and propylene glycol; hydrogenated glucose syrup, triethylcitrate, and acetyltriethyl citrate. Plasticizers are present in the inventive compositions in amounts generally ranging from about 2% to about 20% by weight, in some embodiments from about 5% to about 15% by weight, and in yet other embodiments at about 10% by weight, based on the total weight of the composition. Typically, the ratio of naturally-occurring polypeptide film-former to plasticizer in the compositions of the present invention is from about 6:1 to about 1:1, and in some embodiments about 3:1.

Detackifiers are substances that when combined with the naturally-occurring polypeptide film-former in a liquid or semiliquid composition, i.e., a solution, emulsion, paste, gel, etc., reduce the tackiness or stickiness of the polypeptide. In an embodiment of the cosmetic compositions of the present invention, the detackifier is a solid, for example, a powder, crystal, granule, a fiber, etc. In certain embodiments, the detackifier is a filler, a solid colorant, a fiber, or a mixture thereof. In an embodiment of the present invention, the detackifier is selected from the group consisting of tapioca starch, iron oxide, silica, and combinations thereof.

The detackifier is present in the inventive compositions in amounts generally ranging from about 2% to about 25% by weight, in some embodiments from about 10% to about 20% by weight, and in yet other embodiments at about 14% to 16% by weight, based on the total weight of the composition. In an embodiment of the present invention, the detackifier is a combination of a filler and a pigment, which is present in an amount of about 15% to about 16% by weight, based on the total weight of the composition. Typically, the ratio of naturally-occurring polypeptide film-former to detackifier in the compositions of the present invention is from about 6:1 to about 1:1, and in some embodiments about 2:1.

In certain embodiments, the cosmetic compositions of the present invention contain only naturally-occurring ingredients. In other embodiments, the cosmetic compositions of the present invention are contain only ingredients that meet organic standards.

The compositions of the present invention may contain at least one further (e.g., cosmetically or dermatologically acceptable) ingredient, including additives and adjuvants, such as, for example, anti-foam agents (e.g., simethicone, which is a fluid composition containing polydimethylsiloxane and silica), sunscreen agents, essential oils, fragrances, and cosmetically active agents and dermatological active agents such as, for example, anti-inflammatory agents, defoaming agents, emollients, vitamins, trace elements and essential fatty acids. These ingredients may be soluble or dispersible in whatever phase or phases are present in the cosmetic composition (i.e., aqueous and/or fatty phase).

The compositions for topical application may constitute in particular a cosmetic or dermatological composition for protecting, treating, or caring for keratinous tissue, such as the skin and eye lashes. In certain embodiments, the cosmetic compositions of the present invention are in the form of an eyeliner, mascara, a foundation, a blusher or an eye-shadow, a lipstick, a lipgloss, a care base or balm for the lips, or a concealer product.

In the method of making up or enhancing the appearance of keratinous tissue the present invention, the cosmetic composition is applied to keratinous tissue, such as skin or hair. The cosmetic compositions of the present invention may be applied to keratinous tissue by hand. Alternatively, or in conjunction therewith, they may be applied via an applicator such as a sponge, cotton, brush, or a puff of a natural or synthetic material. In addition, the applicator may be attached to or contained within a container, that serves as a reservoir for the cosmetic composition.

The following examples are intended to further illustrate the present invention. They are not intended to limit the invention in any way. Unless otherwise indicated, all parts are by weight.

### EXAMPLES

### Example 1

A cosmetic composition of the present invention, in the form of mascara, is described below.

| PHASE | INGREDIENTS | AMOUNT (Wt %) |
|---|---|---|
| A | Carnuba Wax | 5 |
| A | Beeswax | 15 |
| A | Organic Argania Spinosa Kernel Oil | 0.1 |
| A | Glyceryl Stearate | 1 |
| A | Sodium Stearoyl Glutamate | 1 |
| A | Cetearyl Alcohol and Cetearyl | 5 |
| | Glucoside | |
| A | Natural Mixture of C12-C16 Fatty | 1 |
| | Alcohols and Vegetal Palmitic | |
| | Acid and Soy Lecithin | |
| A | Silica | 0.5 |
| B | Iron Oxide | 6 |
| C | Water | 51.4 |
| D | Organic Guar Gum | 0.2 |
| E | Xanthan Gum | 0.5 |
| F | Sodium Benzoate | 0.3 |
| G | Sodium Hydroxide (50%) | 0.1 |
| H | Benzyl Alcohol | 0.5 |
| I | Ethanol | 5 |
| I | Water | 2.5 |
| I | Zein | 5 |
| Total | | 100.00 |

The mascara composition described above was prepared as follows. The ingredients of Phase A, i.e., the carnuba wax, beeswax, organic argania spinosa kernel oil, glyceryl stearate, sodium stearoyl glutamate, cetearyl alcohol and cetearyl glucoside, natural mixture of C₁₂-C₁₆ fatty alcohols, vegetal palmitic acid and soy lecithin, and silica were added to a main kettle and heated to 85° C. to melt the waxes. The iron oxide (Phase B) was added to the main kettle and the mixture homogenized for 1 hour at 85° C. Water (Phase C) at 85° C. was added to the main kettle and the mixture was emulsified for 20 minutes. The organic guar gum (Phase D) was added and the mixture mixed for 10 to 15 minutes. The xanthan gum (Phase E) was then added and mixing was continued for an additional 10 minutes. With continued mixing, the mixture was allowed to cool as the sodium benzoate (Phase F) and sodium hydroxide (Phase G) were added to the main kettle. Changing to sweep mixing, ice chips were slowly added to speed cooling, while ensuring that the batch was not shocked to prevent aeration.

The Phase I ingredients, i.e., the ethanol, water and zein, were mixed in a separate kettle to dissolve the zein. At 32° C. to 35° C., the benzyl alcohol (Phase H) and the zein, ethanol, and water solution (Phase I) were added to the main kettle. Mixing was continued until the mixture was homogenous. The composition was removed from the main kettle at room temperature.

### Example 2

The inventive cosmetic composition of Example 1 was compared to a commercial mascara composition, which differs from the inventive composition mainly in that it contains no polypeptide, and to a comparative composition having the same formula as the composition of Example 1, but omitting the polypeptide, e.g., zein, and replacing it with water. The comparisons were from the standpoint of flaking, i.e., wear characteristics.

Flaking was evaluated using the following method. The inventive cosmetic composition of Example 1, a commercially available mascara, and the comparative composition were applied to false eyelashes with 30 strokes with a cosmetic brush, with 3 sets of 10 strokes with a 10 second pause between sets. After 1 hour, the false eyelashes were stroked 30 times with a very stiff brush and 10 times with a conventional cosmetic brush over white paper.

The flaking of each of the mascara compositions, as evidenced by the flakes on the white paper, was scored on a scale of 1 to 5, wherein a score of 1 represented no flaking or almost no flaking; and a score of 5 represented almost complete flaking, which is to say that almost all of the mascara composition had flaked off of the lashes. The mascara composition of the present invention was given a score of 1.0, the commercial mascara was given a score of 3.0, and the comparative composition that omitted zein was given a score of 4.5. The results demonstrate that the inventive composition, containing zein, had much better wear characteristics compared to a mascara not containing a naturally-occurring polypeptide film-former.

### Example 3

A cosmetic composition of the present invention, in the form of an eyeliner, is described below.

| PHASE | INGREDIENTS | AMOUNT (Wt %) |
|---|---|---|
| A | Water | 30.00 |
| A | Zein | 30.00 |
| B | Black Iron Oxide | 10.00 |
| C | Butylene Glycol | 8.00 |
| C | Glycerin | 10.00 |
| D | Guar Gum (Organic) | 0.20 |
| D | Xanthan Gum | 0.50 |
| E | Tapioca Starch | 5.60 |
| F | Ethanol | 5.00 |
| F | Benzyl Alcohol | 0.50 |
| F | Sodium Benzoate | 0.20 |
| Total | | 100.00 |

The cosmetic composition described above is prepared as follows. The ingredients of Phase A, i.e., water and zein, are added to a main kettle at room temperature and dispersed until hydrated and uniform. The iron oxide (Phase B) is then added to the main kettle and the mixture dispersed for 30 minutes while maintaining at room temperature. Phase C (butylene glycol and glycerin) is then added to the kettle and dispersed until uniform. Phase D (guar gum and xanthan gum) is then added to the kettle and mixed until completely dispersed while maintaining at room temperature. Phase E (tapioca starch) is added to the kettle and mixed until uniform while maintaining at room temperature. Phase F (ethanol, benzyl alcohol, and sodium benzoate) is added to the kettle and mixed until uniform while maintaining at room temperature.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising a naturally-occurring polypeptide film-former, a thickener, and a solvent.

2. The cosmetic composition of claim 1, wherein said polypeptide is plant-derived.

3. The cosmetic composition of claim 2, wherein said polypeptide is a prolamine.

4. The cosmetic composition of claim 3, wherein said prolamine is zein.

5. The cosmetic composition of claim 3, wherein said prolamine is gliadin.

6. The cosmetic composition of claim 3, wherein said prolamine is hordein.

7. The cosmetic composition of claim 4, wherein said solvent comprises a lower alkanol.

8. The cosmetic composition of claim 7, wherein said solvent comprises ethanol.

9. The cosmetic composition of claim 1, wherein said thickener is selected from the group consisting of guar gum, xanthan gum, and mixtures thereof.

10. The cosmetic composition of claim 1, further comprising a wax, an oil, an emulsifier, a dispersion enhancing agent, a colorant, a preservative, a pH adjuster, a filler, and water.

11. The cosmetic composition of claim 10, wherein said wax is selected from the group consisting of carnauba wax, beeswax, and mixtures thereof.

12. The cosmetic composition of claim 10, wherein said oil is argania spinosa kernel oil.

13. The cosmetic composition of claim 10, wherein said emulsifier is selected from the group consisting of glyceryl stearate, sodium stearoyl glutamate, cetearyl alcohol, cetearyl glucoside, and mixtures thereof.

14. The cosmetic composition of claim 10, wherein said dispersion enhancing agent is selected from the group consisting of C₁₂-C₁₆ fatty alcohols, palmitic acid, lecithin, and mixtures thereof.

15. The cosmetic composition of claim 10, wherein said colorant is iron oxide.

16. The cosmetic composition of claim 10, wherein said preservative is selected from the group consisting of sodium benzoate, benzyl alcohol, and mixtures thereof.

17. The cosmetic composition of claim 10, wherein said pH adjuster is sodium hydroxide.

18. The cosmetic composition of claim 10, wherein said filler is silica.

19. The cosmetic composition of claim 1, further comprising a colorant, a preservative, and water.

20. A package comprising a container having disposed therein the composition of claim 1, and optionally an applicator for applying the composition to keratinous tissue.

21. A method of making up or enhancing the appearance of keratinous tissue, comprising applying to keratinous tissue a cosmetic composition comprising a naturally-occurring polypeptide film-former, a thickener, and a solvent.

22. A method of making a cosmetic composition comprising mixing together a naturally-occurring polypeptide film-former, a thickener, and a solvent.
